(19) United States
(12) Patent Application Publication
(10) Pub. No.: US 2020/0170905 A1
(43) Pub. Date: Jun. 4, 2020

(54) KIT FOR CLEANSING AND REFRESHING THE HAIR AND THE SCALP

(71) Applicant: The Procter & Gamble Company, Cincinnati, OH (US)

(72) Inventors: Supriya Punyani, Singapore (SG); Ioannis Constantine Constantinides, Wyoming, OH (US); Steven Hardy Page, Lawrenceburg, IN (US); Sandra Nichole Isaacs, Colerain, OH (US)

(21) Appl. No.: 16/209,230

(22) Filed: Dec. 4, 2018

(51) Int. Cl. A61K 8/34; A61K 8/39; A61K 8/86; A61Q 5/02

(52) U.S. Cl. CPC A61K 8/345; A61K 8/39; A61K 8/342; A61K 8/0275; A61Q 5/02; A61K 2800/88; A61K 2800/87; A61K 8/86

(57) ABSTRACT

The present invention is directed to kit for cleansing and refreshing the hair and the scalp comprising (1) a leave-on aqueous cleansing composition comprising (a) a 1,2-diol having a carbon chain with a length of more than 8 carbons in its molecule, and (b) hydrophobic particles, and (2) a substrate for partially removing the leave-on aqueous cleansing composition from the hair and the scalp. The present invention is also directed to a method of cleansing and refreshing the hair and the scalp by applying a leave-on aqueous cleansing composition on the hair and the scalp and partially removing the composition using the substrate.

### FIELD OF THE INVENTION

The present invention relates to a kit for cleansing and refreshing the hair and the scalp comprising (1) a leave-on aqueous cleansing composition comprising a 1,2-diol and hydrophobic particles, and (2) a substrate for partially removing the leave-on aqueous cleansing composition from the hair and the scalp. The present invention also relates to a method of cleansing and refreshing the hair and the scalp comprising the steps of (a) applying a leave-on aqueous cleansing composition to the hair and the scalp, and (b) partially removing the leave-on aqueous cleansing composition from the hair and the scalp using a substrate.

### BACKGROUND OF THE INVENTION

Clean Scalp and Hair is described by consumers as having no sticky or greasy feel, no clumped fibers, no odor, and no hair weigh-down. Generally, consumers perceive unclean scalp and hair when sebum in liquid state builds upon their scalp and their hair during the end of the day. The liquid sebum on the hair and the scalp is often associated with unclean, greasy, oily and dirty look, feel and smell. Sebum is continuously secreted out of the sebaceous glands on the scalp in liquid form. Due to dynamic environment (exposure to UV and microflora), sebum is unstable and its composition rapidly changes. As a result, it typically exists in more than one phase on the scalp and it is transferred to the hair during the day. Cleansing with surfactant-containing shampoo, removed approximately 60-90% of the accumulated sebum, depending on the surfactant concentration. Most of the shampoos remove on average 80% of the sebum. However, as sebum secretion is a continuous process, sebum re-appears on the scalp in significant amounts, accumulating on it within 5-6 hours after shampooing. As mentioned above, sebum gets progressively transferred to the hair fibers, which leads to unclean consumer perception within 5-6 hours from the previous wash. Most consumers use surfactant-containing shampoos to clean their hair, whereas there is a minority of consumers who use oil-based products to clean their hair. It is perceived that many shampoos with high surfactant content strip the hair fiber surface from natural lubricants, leading to dry and squeaky hair feel. Thus, there is an undesired trade-off in shampooing and a need exists for hair care products that can (a) effectively delay the appearance and feel of unclean scalp and hair and (b) remove sebum without the above-mentioned negative trade-offs. The present invention has surprisingly found that this can be achieved by the use of a cleansing kit comprising (1) a leave-on aqueous cleansing composition comprising a 1,2-diol and hydrophobic particles, and (2) a substrate for partially removing the leave-on aqueous cleansing composition from the hair and the scalp.

Without wishing to be bounded by theory, the use of the leave-on aqueous cleansing composition comprising the combination of these materials achieves the benefits by modifying the sebum physical properties, such as its melting characteristic, and/or by absorbing the sebum. The substrate enhances the process by potentially enabling both the application of the composition on the hair and the scalp, the intimate contact between the components of the leave-on aqueous cleansing composition and the sebum, and by partially removing the sebum from the hair and the scalp. As a result, the amount of sebum is reduced and also the transfer of the remaining sebum from the scalp to the hair fibers is reduced, making hair appearance and feel to be less unclean. In addition, the cleansing kit and the corresponding method of cleansing the hair and the scalp enables an efficient, effective and durable cleansing and refreshing of the hair and the scalp without requiring the use of rinsing with water in a shower. The process is relatively convenient and can be performed by the consumer at any location and at any time during the day.

### SUMMARY OF THE INVENTION

The present invention is directed to a kit for cleansing and refreshing the hair and the scalp comprising
a. a leave-on aqueous cleansing composition comprising
   (1) from about 0.1 wt. % to about 12 wt. % of a 1,2-diol having a carbon chain with a length of more than 8 carbons;
   (2) from about 0.1 wt. % to about 10 wt. % of a solid particle, wherein
      (a) the interfacial tension between the solid particle and sebum is from about 5 to about 18 dyn/cm;
      (b) the sebum exhibits spreading coefficient on the solid, which is greater than about 22 dyn/cm; and
      (c) the work of adhesion of the sebum to the solid particle, which is greater than about 75 dyn/cm.
   (3) an aqueous carrier; and
b. a substrate for partially removing the leave-on aqueous cleansing composition from the hair and the scalp.

The kit for cleansing and refreshing the hair and the scalp of the present invention provides an efficient, effective and durable cleansing and refreshing of the hair and the scalp without requiring the use of rinsing with water in a shower.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Dermatologically acceptable” or “cosmetically acceptable” means that the compositions or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Safe and effective amount” means an amount of a compound or composition sufficient to significantly induce a positive benefit.

“Leave-on aqueous cleansing composition” in reference to the present invention is a composition that is intended to be applied to and allowed to remain on the keratinous tissue, although a portion of it is removed by the substrate. The aqueous cleansing composition is to be distinguished from compositions, which are applied to the hair and subsequently (in a few minutes or less) removed either by washing, and/or rinsing. Thus, the aqueous cleansing composition of the present invention is not a composition used in rinse-off applications such as shampoos, rinse-off conditioners, facial cleansers, hand cleansers, body wash, or body cleansers. The leave-on cleansing composition of the present invention may be substantially free of cleansing or detersive surfactants. For example, the leave-on aqueous cleansing compositions may be left on the keratinous tissue for at least 15 minutes. For example, the leave-on aqueous cleansing composition may comprise (a) less than 1% detersive surfactants, (b) less than 0.5% detersive surfactants, or (c) 0% detersive surfactants. The leave-on aqueous cleansing composition may, however, contain emulsifying, dispersing or other processing surfactants that are not intended to provide any significant cleansing benefits when applied topically to the hair.

“Apply” or “application” as used in reference to a composition, means to apply or spread the composition of onto a keratinous tissue, such as the hair and/or the scalp. The leave-on aqueous cleansing composition of the present invention can be applied to and remain on the hair and the scalp, although some portion of it is removed by the substrate.

The leave-on aqueous cleansing composition can be applied by transferring the composition from its container (a) by spraying onto the hair and/or scalp via a spraying device, (b) by applying directly onto the hair and/or scalp via the container as a liquid (c) by applying onto the hair and/or scalp via an applicator as a liquid, and (d) by applying onto the hands and then onto the hair and/or the scalp by the hands. The composition may be spread onto the hair and the scalp after the initial transfer using the hands or an applicator. Then, a substrate is used to further spread the leave-on cleansing composition onto the hair and/or scalp that partially removes the leave-on cleansing composition form the hair and/or the scalp.

Alternatively, the leave-on aqueous cleansing composition can be applied onto a substrate and then transferred to the hair and/or the scalp from the substrate. The substrate spreads the leave-on aqueous cleansing composition onto the hair and scalp. Optionally, another substrate can be then used to further remove additional amount of the leave-on aqueous cleansing composition onto the hair and scalp. Another option is to use one substrate that comprises two regions A and B; region A can be used for applying the leave-on aqueous cleaning composition on and for spreading the composition onto the hair and/or the scalp and region B can be used for partially removing the composition form the hair and/or the scalp.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography. “QS” means sufficient quantity for 100%.

Unless otherwise specified, the term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Solid Particles”, as used herein, means particle and/or powder blends that may be free flowing compositions or suspensions of synthetic porous agglomerates comprising of organic and/or inorganic compounds.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

“Substrate”, as used herein, means a water-insoluble substrate, which comprises single-layer or multi-layer sheet-form materials. Besides paper tissues, corresponding tissue cloths made from fibers or nonwovens may also be used. Examples of natural fibers include silk, cellulose, keratin, wool, cotton, jute, linen, flax; examples of synthetic fibers include acetate, acrylate, cellulose ester, polyamide, polyester, polyolefin, polyvinyl alcohol, polyurethane fibers or even additive-hydrophilized woven polyolefin fabrics and blends of these fibers or woven fabrics.

The method of cleaning and refreshing the hair and/or the scalp of the present invention using kit for cleansing and refreshing the hair and the scalp provides longevity of hair clean feel and appearance. It also provides excellent cleaning performance without significantly negatively affecting hair feel. The benefit is partially enabled by the leave-on aqueous cleansing composition, which comprises a combination of ingredients that modify sebum. This ingredient combination (a) can modify sebum properties such as its melting characteristics and/or (b) can absorb sebum, so that sebum can be restrained from transferring from the scalp to the hair fibers over time. The benefit is also enabled by the substrate, which enables the partial removal of the sebum. The sebum removal is shown by measuring the sebum content of scalp or hair that is exposed to the cleansing method using the cleansing kit of the present invention compared to the sebum content of scalp or hair that is exposed to a cleansing methods (1) and (2), wherein (1) is the corresponding method which uses control aqueous compositions having no sebum modifying ingredients (1,2-diol and (2) is the corresponding method which does not include a substrate.

### Components of the Kit for Cleansing

The present invention is directed to a kit for cleansing and refreshing the hair and the scalp comprising:
a. a leave-on aqueous cleansing composition comprising
   (1) from about 0.1 wt. % to about 12 wt. % of a 1,2-diol having a carbon chain with a length of more than 8 carbons;
   (2) from about 0.1 wt. % to about 10 wt. % of a solid particle, wherein
      (a) the interfacial tension between the solid particle and sebum is from about 5 to about 18 dyn/cm;
      (b) the sebum exhibits spreading coefficient on the solid, which is greater than about 22 dyn/cm; and
      (c) the work of adhesion of the sebum to the solid particle, which is greater than about 75 dyn/cm.
   (3) an aqueous carrier; and
b. a substrate for partially removing the leave-on aqueous cleansing composition from the hair and the scalp.

### A. Leave-On Aqueous Cleansing Composition

The combination of the 1,2-diols and solid particles can (a) modify the sebum physical properties such as melting characteristic and (b) absorb the sebum. The leave-on aqueous cleansing composition can be in the form of a liquid of various viscosity values. If the high shear viscosity is low, it can be delivered as a spray. In all cases, the low shear viscosity of the composition must be relatively high in order to be phase stable, as the composition contains particles that can settle or come to the top.

Ingredients of the Leave-On Aqueous Cleansing Composition

### 1. 1,2-Diol (for Modification of the Melting Characteristics of Sebum)

The leave-on aqueous cleansing composition of the present invention may comprise 1,2-diol in a concentration range from about 0.1% to about 12%; from about 0.2 to about 5%; from about 0.5 to about 4%; and from about 1.0% to about 3.0% by weight of the leave-on aqueous cleansing composition. The 1,2-diols in the leave-on aqueous cleansing composition have a carbon chain with length of more than 8 carbons. Non-limiting examples are 1,2-diols include 1,2-dodecnediol, 1,2-decanediol, 1,2-octadecanediol. Without being limited by theory, such 1,2-diols contribute to the modification of the melting characteristics of sebum, making it more available to be absorbed/adsorbed by the solid which is present in the composition.

In an attempt, to identify materials that can modify the physical characteristics of sebum, such as their melting temperature, various mixtures of materials with sebum (in a 1:1 weight ratio) are prepared and their melting characteristics of the mixtures are measured using differential scanning calorimetry (DSC). The DSC method that is used is described in detail in the “Evaluation Methods” section. The results following table 1 provides the corresponding DSC measurements.

TABLE 1
Melting characteristics of mixtures of various substances with sebum

| | Endothermic | Endothermic | Endothermic | Endothermic |
|---|---|---|---|---|
| Sebum Modifier | Temperature | Temperature | Temperature | Temperature |
| Material | Peak 1 (° C.) | Peak 2 (° C.) | Peak 3 (° C.) | Peak 4 (° C.) |
| Petrolatum | −23.00 | 3.00 | 16 | 40 |
| 1,2- | −18.00 | 4.90 | 21 | 36 |
| dodecanediol | | | | |
| Stearyl alcohol | −18.00 | 6.00 | 15 | 36 |
| Cetyl alcohol | −18.00 | 4.00 | 18 | 26 |
| 1,2-Decanediol | −20.00 | | | 42 |
| Stearic acid | −18.20 | 6.50 | | 39 |
| Zinc carbonate | 0.5 | 7 | | 75 |
| Zinc stearate | −4 | 8 | 21 | 97 |
| 1,2- | −4 | 10 | 20 | 51 |
| Octadecanediol | | | | |

### 2. Hydrophobic Solid Particles (for Abdsorbing Sebum)

The leave-on aqueous cleansing composition of the present invention may comprise solid particles in a concentration range from about 0.1% to about 10%; from about 0.5% to about 5%; from about 1.0% to about 2.0% by weight; and a further embodiment from about 1.0 to about 2.0% by weight of the leave-on aqueous cleansing composition.

The particle surface of the particles that are present in the leave-on aqueous cleansing composition have the following properties
(a) the interfacial tension between the solid particle and sebum is from about 5 to about 18 dyn/cm;
(b) the sebum exhibits spreading coefficient on the solid, which is greater than about 22 dyn/cm; and
(c) the work of adhesion of the sebum to the solid particle, which is greater than about 75 dyn/cm.

Non-limiting examples of hydrophobic solid particle or hydrophobically modified solid particle particles that are present in the leave-on aqueous cleansing composition are silica silylate, zinc carbonate, hydrophobic clay, zinc oxide, polyethylene powders, polypropylene powders, polystyrene powders, calcium silicate, polyethylene, nylon, boron nitride, mica, clays such as bentonite, montmorillonite and kaolin, zeolite, cyclodextrins, fumed silica, synthetic clays such as polymer powders including natural, synthetic, and semisynthetic cellulose, fluorocarbon resins, polypropylene, modified starches of cellulose acetate, particulate cross-linked hydrophobic acrylate or methacrylate copolymers and mixtures thereof. In an embodiment, solid particles for the leave-on aqueous cleansing composition are silica silylate, salicylic acid, 2,4-dihydroxy benzoic acid, 4-chlororesorcinol, 1,2,4-Trihydroxybenzene and zinc carbonate.

Other non-limiting examples of hydrophobic solid particle or hydrophobically modified solid particle particles that are present in the leave-on aqueous cleansing composition include starches hydrophobically modified to have a high capacity for absorbing oils; such starches can be modified with alkyl or alkenyl substituted dicarboxylic acids; such materials may contain counter-ions, for example metals such as aluminum. A non-limiting example of such material is Natrasorb HFB available from National Starch and Chemical Company, U.S.A., which contains aluminum starch octenyl succinate. Other suitable materials from National Starch and Chemical include Natrasorb Bath, Dry-Flow PC, Dry-How XT, and Dry-Flow Pure.

Other non-limiting examples of hydrophobic solid particle or hydrophobically modified solid particle particles that are present in the leave-on aqueous cleansing composition include modified proteins such as Vegepol (sodium C8-16 isoalkylsuccinyl soy protein succinate) from Brooks Industries, N.J., and the like.

In order to identify solid particles that can effectively absorb sebum, the absorption/adsorption characteristics of sebum on various solid particles are measured using the contact angle method, which is described in the “Evaluation Method” section. The following table provides the corresponding measurements.

TABLE 2
Measurements of sebum absorption/adsorption on various solids

| | | | Total | | | Inter- |
|---|---|---|---|---|---|---|
| | | | (Polar + | Work of | Spreading | facial |
| | Non- | | Non- | Adhesion | Coefficient | Tension |
| Material | polar | Polar | polar) | (sebum) | (sebum) | (sebum) |
| Talc | 22.53 | 5.91 | 28.44 | 55.01 | 1.07 | 0.40 |
| Silica | 32.87 | 32.20 | 65.08 | 76.37 | 22.43 | 15.68 |
| Silica | 49.07 | 26.14 | 75.21 | 86.71 | 32.77 | 15.47 |
| silylate | | | | | | |
| (hydro- | | | | | | |
| phobic | | | | | | |
| silica) | | | | | | |
| Cellulose- | 27.50 | 41.00 | 68.50 | 74.24 | 20.30 | 21.23 |
| untreated | | | | | | |
| Zinc | 44.83 | 26.38 | 71.21 | 83.78 | 29.84 | 14.40 |
| Carbonate | | | | | | |

### 3. Other Components in the Leave-On Aqueous Cleansing Composition

The leave-on aqueous cleansing composition can be delivered as a spray directly on the hair or it can be sprayed on the substrate and the applied to the hair and scalp.

The leave-on aqueous cleansing composition may comprise one or more detersive surfactant. In addition, it may comprise other optional ingredients such as silicone or organic conditioning agents, hair health actives, anti-dandruff actives, and other ingredients.

a. Aqueous Carrier

The leave-on aqueous cleansing composition of the present invention comprises and aqueous carrier. Accordingly, the composition may comprise water from about 78% to about 99.5%; from about 90% to about 99%; from about 95% to about 98% by weight of the leave-on aqueous cleansing composition. The aqueous carrier may also comprise water miscible solvent or mixture of solvents. Non-limiting examples of water-miscible solvents include lower alkyl alcohols and polyhydric alcohols. The lower alkyl alcohols useful herein are monohydric alcohols having 1 to 6 carbons, such as ethanol and isopropanol. The polyhydric alcohols useful herein include propylene glycol, dipropylene glycol, hexylene glycol, glycerin, and propane diol. 1,3-butanediol. The ratio of water-miscible solvent to water may be form about 0 to about 2; from about 0 to about 0.1; or from about 0 to about 0.01.

b. Rheology Modifier

The leave-on aqueous cleansing composition of the present invention may comprise one or more rheology modifier to provide phase stability and/or improved in-use consumer experience. Any suitable rheology modifier can be used. The leave-on aqueous cleansing composition may comprise from about 0.05% to about 5% of a rheology modifier; from about 0.1% to about 3% of a rheology modifier; or from about 0.5% to about 1% of a rheology modifier by weight of the leave-on aqueous cleansing composition.

The rheology modifier may be a polymeric rheology modifier such as polyacrylamide. The polymeric rheology modifier may be a homopolymer based on acrylic acid, methacrylic acid or other related derivatives. Non-limiting examples include polyacrylate, polymethacrylate, polyethylacrylate, and polyacrylamide. The rheology modifier may also be alkali swellable or hydrophobically-modified alkali swellable acrylic copolymers or methacrylate copolymers non-limiting examples include acrylic acid/acrylonitrogens copolymer, acrylates/steareth-20 itaconate copolymer, acrylates/ceteth-20 itaconate copolymer, acrylates/aminoacrylates copolymer, acrylates/steareth-20 methacrylate copolymer, acrylates/beheneth-25 methacrylate copolymer, acrylates/steareth-20 methacrylate cross-polymer, acrylates/vinylneodecanoate crosspolymer, and acrylates/C10-C30 alkyl acrylate cross-polymer. The rheology modifier may be a soluble cross-linked acrylic polymer; a non-limiting example of this class includes carbomers. The rheology modifier may be a alginic acid-based material, non-limiting examples of which include sodium alginate, and alginic acid propylene glycol esters. The rheology modifier may be an associative polymeric thickeners, non-limiting examples of which include hydrophobically modified cellulose derivatives; hydrophobically modified alkoxylated urethane polymers, nonlimiting example include PEG-150/decyl alcohol/SMDI copolymer, PEG-150/stearyl alcohol/SMDI copolymer, polyurethane-39; hydrophobically modified, alkali swellable emulsions, non-limiting examples include hydrophobically modified polypolyacrylates, hydrophobically modified polyacrylic acids, and hydrophobically modified polyacrylamides; hydrophobically modified polyethers wherein these materials may have a hydrophobe that can be selected from cetyl, stearyl, oleayl, and combinations thereof, and a hydrophilic portion of repeating ethylene oxide groups with repeat units from about 10 to about 300; from about 30 to about 200; or from about 40 to about 150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate. The rheology modifier may be cellulose and derivative, non-limiting examples of which include microcrystalline cellulose, carboxymethylcelluloses, hydroxyethylcellulose, hydroxypropylcellulose, hydroxypropylmethylcellulose, methylcellulose, ethyl cellulose, nitro cellulose, cellulose sulfate, cellulose powder, and hydrophobically modified cellulose. The rheology modifier may be a guar or guar derivative, non-limiting examples of which include hydroxypropyl guar, and hydroxypropyl guar hydroxypropyl trimonium chloride. The rheology modifier may be polyethylene oxide; polypropyne oxide or POE-PPO copolymer. The rheology modifier may be polyvinylpyrrolidone, cross-linked polyvinylpyrrolidone or derivative or polyvinyalcohol or its derivative or polyethyleneimine or its derivative. The rheology modifier may be silica, non-limiting examples of which include fumed silica, precipitated silica, and silicone-surface treated silica. The rheology modifier may be water-swellable clay, non-limiting examples of which include laponite, bentolite, montmorilonite, smectite, and hectonite. The rheology modifier may be a gum, non-limiting examples of which include xanthan gum, guar gum, hydroxypropyl guar gum, Arabia gum, tragacanth, galactan, carob gum, karaya gum, and locust bean gum. The rheology modifier may be dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of other rheology modifiers include acrylamide/ammonium acrylate copolymer (and) polyisobutene (and) polysorbate 20, acrylamide/sodium acryloyldimethyl taurate copolymer/isohexadecane/polysorbate 80, acrylates copolymer, acrylates/beheneth-25 methacrylate copolymer, acrylates/C10-C30 alkyl acrylate cross-polymer, acrylates/steareth-20 itaconate copolymer, ammonium polyacrylate/isohexadecane/PEG-40 castor oil, C12-16 alkyl PEG-2 hydroxypropylhydroxyethyl ethylcellulose (HM-EHEC), carbomer, cross-linked polyvinylpyrrolidone (PVP), dibenzylidene sorbitol, hydroxyethyl ethylcellulose (EHEC), hydroxypropyl methylcellulose (HPMC), hydroxypropyl methylcellulose (HPMC), hydroxypropylcellulose (HPC), methylcellulose (MC), methylhydroxyethyl cellulose (MEHEC), PEG-150/decyl alcohol/SMDI copolymer, PEG-150/stearyl alcohol/SMDI copolymer, polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, polyurethane-39, sodium acrylate/acryloyldimethyltaurate/dimethylacrylamide, crosspolymer (and) isohexadecane (and) polysorbate 60, sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol Ulterez 30, Carbopol 1342, Sepigel™ 305, Simulgel™600, Sepimax Zen, and combinations thereof.

c. Structurants

The leave-on aqueous cleansing composition of the present invention may comprise structurants or suspending agents including crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents. Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine Still other suitable suspending agents include di(hydrogenated tallow)phthalic acid amide, and crosslinked maleic anhydride-methyl vinyl ether copolymer.

d. Emulsifiers

The 1,2-diol may be added into the leave-on aqueous cleansing composition neat or as pre-emulsion. In the latter case, an emulsifier is used to make the pre-emulsion. The emulsifier selection is guided by the Hydrophilic-Lipophilic-Balance value (HLB value) of emulsifiers. Suitable range of HLB value is from about 6 to about 16; or from about 8 to about 14. Emulsifiers with an HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers. Non-limiting examples of nonionic emulsifiers are laureth-7 and PEG-100 stearate.

The concentration of the emulsifier in the composition should be sufficient to provide the desired emulsification of the conditioning actives to achieve desired particle sizes and emulsion stability, and generally ranges from about 0.1 wt % to about 5 wt. %, from about 0.5 wt. % to about 3 wt. %, from about 0.6 wt. % to about 2 wt. % by weight of the composition.

e. Solvents

The leave-on aqueous cleansing composition of the present invention may comprise one or more solvents that are not water-miscible, non-limiting examples include isopropyl myristate, 2-hexyldecanol, PEG-3 glyceryl Cocoate, PEG-7 glyceryl cocoate, isododecane and isoparaffin C11-12 and mixtures thereof. The concentration of the solvent in the composition may range from about 0.1 wt % to about 20 wt. %, from about 0.5 wt. % to about 10 wt. %, from about 1 wt % to about 5 wt. % by weight of the composition.

f. Detersive Surfactants

The leave-on aqueous cleansing composition of the present invention may also comprise one or more detersive surfactants. The detersive surfactants may be anionic, amphoteric, nonionic, cationic or mixtures thereof. The concentration of the detersive surfactant in the composition may range from about 0.1 wt % to about 10 wt. %, from about 0.5 wt. % to about 10 wt. %, from about 1 wt. % to about 5 wt. % by weight of the composition.

g. pH of the Composition

The leave-on aqueous cleansing composition of the present invention may also comprise one or more pH adjusting material. The composition may have a pH in the range from about 2 to about 10, at 25° C.; in the range from about 2 to about 7; or in the range of 3.5 to about 6.5. Alternatively, the composition may have a pH in the range from about 3.5 to 6 or in the range from about 5.25 to about 7. The leave-on aqueous cleansing composition of the present invention may further comprise one or more pH buffering agent. Examples of buffering agents are well known in the art and include, for example, ammonia/ammonium acetate mixture and monoethanolamine (MEA), citric acid and citrate salt.

h. Conditioning Benefit Agents

The leave-on aqueous cleansing composition of the present invention may also comprise one or more conditioning benefit agent, such as silicones or organic hydrophobic oils. The conditioning agents that are oils should be added in moderate quantities, for example, less than 3 wt % by weight of the leave-on aqueous cleansing composition, in order to preserve the absorbing ability of the solid particles of the composition leave-on aqueous cleansing composition. The concentration of the conditioning benefit in the composition may range from about 0 wt % to about 3 wt. %, from about 0 wt. % to about 1 wt. %, from about 0 wt. % to about 0.5 wt. % by weight of the composition.

i. Other Ingredients

The leave-on aqueous cleansing composition of the present invention may also comprise one or more other benefit agents or other ingredients such anti-dandruff agents, anti-itch agents, other scalp health agents, anti-oxidants, vitamins, chelation agents, sensates, colorants, bleaching agents, preservatives, perfumes, humectants and mixtures thereof.

Particularly important is the presence of perfumes and/or sensates. Such ingredients may provide sensorial signals to the consumer during and after the cleansing method that the hair and/or the scalp is clean and refreshed.

### B. Substrate

The substrate of the kit for cleansing and refreshing the hair and the scalp is water-insoluble, which means that it will not dissolve or readily break apart upon contact with an aqueous carrier at 25° C. The substrate may comprise single-layer or multi-layer sheet-form materials. Besides paper tissues, corresponding tissue cloths made from fibers or nonwovens may also be used. Examples of natural fibers include silk, cellulose, keratin, wool, cotton, jute, linen, flax; examples of synthetic fibers include acetate, acrylate, cellulose ester, polyamide, polyester, polyolefin, polyvinyl alcohol, polyurethane fibers or even additive-hydrophilized woven polyolefin fabrics and blends of these fibers or woven fabrics. The substrate of the kit for cleansing and refreshing the hair and the scalp may be a sheet having rectangular or any other shape having surface area from about 50 cm² to about 300 cm²; or from about 100 cm² to about 200 cm². The substrate may be porous. It must have the ability to absorb the leave-on aqueous cleansing composition and partially remove it from the hair and/or the scalp.

The substrate may be used just once and disposed. Alternatively, the substrate may be retained, rinsed and dried for multiple uses.

The kit for cleansing and refreshing the hair and the scalp may be sold in stores or via the internet as a set of one or more containers of the leave-on aqueous cleansing composition along with a number of substrates.

Method of Cleansing the Hair or the Scalp

The present invention also relates to a method of cleansing and refreshing the hair and the scalp comprising the following steps:
(a) applying a leave-on aqueous cleansing composition to the hair and the scalp, and
(b) partially removing the leave-on aqueous cleansing composition from the hair and the scalp using a substrate.
   a. Step 1: Application of the Leave-On Aqueous Cleansing Composition onto the Hair and the Scalp

The consumer may apply the leave-on aqueous cleansing composition onto the hair and/or the scalp by the following methods:
(1) Spraying the composition directly onto the hair and/or the scalp using a spray device, such as aerosol spray, mechanical spray or squeeze spray; the spraying step may be followed by further spreading of the composition onto the hair and/or the scalp by the hands of the consumer or by another applicator device, before the substrate is used to partially remove the composition from the hair and/or the scalp.
(2) directly dispensing the composition in liquid form onto the hair and/or the scalp from its container having one or more nozzles or any other relevant applicator attached to the container (a) by decanting/tilting the container by using gravity or (b) by a squeeze bottle; the direct dispensing step may be followed by further spreading of the composition onto the hair and/or the scalp by the hands of the consumer or by another applicator device, before the substrate is used to partially remove the composition from the hair and/or the scalp;
(3) directly dispensing the composition in a foam form from its container onto the hair and/or the scalp via an aerosol dispenser or a mechanical foam dispenser (pump or trigger or squeeze); the direct dispensing step of the composition in a foam form may be followed by further spreading of the composition onto the hair and/or the scalp by the hands of the consumer or by another applicator device, before the substrate is used to partially remove the composition from the hair and/or the scalp.
(4) dispensing the composition from its container by spraying, by decanting or by squeezing the container onto the hands and transferring and spreading the composition onto the hair and/or the scalp using the hands;
(5) dispensing the composition in a foam form from its container onto the hands and transferring and spreading the composition onto the hair and/or the scalp using the hands;
(6) dispensing the composition from its container by spraying, by decanting or by squeezing the container into an applicator system, such as a bowl and a brush, and transferring and spreading the composition onto the hair and/or the scalp using the applicator system;
(7) dispensing the composition in a foam form from its container into an applicator system, such as a bowl and a brush, and then transferring and spreading the product onto the hair and/or the scalp using the applicator system;
(8) Spraying the composition onto the substrate using a spray device, such as aerosol spray, mechanical spray or squeeze spray) and then transferring and spreading the composition onto the hair and/or the scalp using the substrate;
(9) dispensing the composition in liquid form onto the substrate from its container (a) by decanting/tilting the container by using gravity or (b) by a squeeze bottle and then transferring and spreading the composition onto the hair and/or the scalp using the substrate;
(10) dispensing the composition in a foam form from its container onto the substrate and transferring and spreading the composition onto the hair and/or the scalp using the substrate
   b. Step 2: Partial Removal of the Leave-On Aqueous Composition from the Hair and/or the Scalp

After the application of the leave-on aqueous cleansing composition onto the hair and/or the scalp, the cleansing method of the present invention comprises the partial removal of the composition from the hair and/or the scalp with a substrate. The substrate has the ability to absorb some of the composition, thus, partially removing the applied composition from the scalp and/or the hair, removing with it sebum, and cleansing and refreshing the hair and/or scalp of the consumer.

In the case of the application methods (1) to (8) described in the previous paragraph, the step of applying the composition on the hair and/or the scalp is followed by partially removing the composition by massaging the hair and/or the scalp by the hand holding the substrate. This step also contributes to a better spreading of the composition on the hair and/or the scalp, also contributing to a more effective modification of the physical characteristics of the sebum and a more effective absorption of the sebum by the solid particles of the composition. Optionally, the step of partially removing the composition from the hair and/or the scalp may be repeated using a second piece of substrate for removing an additional quantity of the composition, and, thus, removing an additional amount of sebum from the hair and/or the scalp.

In the case of the application methods (9) and (10) described in the previous paragraph, the step of partial removing of the composition is inherently performed by the completion of the step described above. Optionally, the step of applying the composition onto the hair and the scalp using the composition on the substrate and partially removing the composition may be followed by massaging the hair and/or the scalp with another substrate unit to further remove additional amount of the leave-on aqueous cleansing composition from the hair and scalp. Another option is to use one substrate that comprises two regions A and B; region A can be used for applying the leave-on aqueous cleaning composition and for spreading the composition onto the hair and/or the scalp and region B can be used for partially removing the composition form the hair and/or the scalp.

Step 2 may be performed immediately after step 1, or between 1 second and 1 minute after step 1, or between 1 minute and 1 hour after step 1, or between 1 to 24 hours after step 1, or between 1 and 3 days after step 1. A water rinsing step may be performed between step 1 and step 2.

### Evaluation Methods

A. Sample Preparation for Differential Scanning Calorimetry (DSC) Measurement

An amount of 1.0 g of artificial sebum (Table 3) is mixed with 1.0 g of sebum modifier material at room temperature and heated at 40° C. water bath for 2 minutes to a uniform solution/mixture and the solution/mixture is cooled at room temperature. The melting characteristics of the prepared sample are determined using the differential scanning calorimetry (DSC) method as described below.

TABLE 3
Artificial Sebum Composition

| Raw Material | Weight % |
|---|---|
| Stearic Acid | 14 |
| Oleic acid | 8 |
| Squalene | 12 |
| Cetyl Palmitate | 12 |
| Isostearyl Isostearate | 12 |
| Trioctanoin | 20 |
| Caprylic Capric | 20 |
| Triglyceride | |
| Cholesterol | 2 |

B. Differential Scanning Calorimetry (DSC) Measurement

A DSC 204 Netzsch TASC 414/3A is used for the tests, which are performed in triplicate samples. The samples consist of approximately 5.5 mg of a 1:1 uniform solution/mixture combination of artificial sebum and sebum modifier. The sample is placed into T-Zero aluminum DSC pans, and then covered with stainless steel mesh A sand baseline and burn-off is performed before running samples, and after every 10 samples. The tests are conducted within the temperature range of −50° C. to 300° C. at 5° C./min under 200 ml/min nitrogen purge. An empty pan of the same type is employed as a reference and tested under the same experimental conditions. The standard deviation of this method is less than 5%. Temperature measurements are taken at the endotherm peaks of physical changes in sebum components and curve-fitting energy integration is performed for the entire endotherm.

C. Measurement of Solid Particles Physical Properties using Contact Angle Method:

The contact angle on solid particles is determined using a modified ASTM D7490-13, titled “Standard Test Method for Measurement of the Surface Tension of Solid Coatings, Substrates and Pigments using Contact Angle Measurements”.

The contact angle on both sides of a drop of distilled water is measured on the finished side of the clean untreated ceramic tile. The contact angle measurement is repeated for a drop of diidomethane (supplied by Sigma Aldrich, St. Louis, Mo.). The two contact angle values (for water and diidomethane) are then substituted into two separate expressions of the Owens-Wendt-Kaelble equation (one for each liquid). This results in three equations and two unknowns, which are then solved for the dispersion and polar components of surface tension.

Equipment used for Contact Angle Method

Goniometer—An instrument consisting of a controlled light source, a stage to hold the tile, and a microscope or camera for viewing of the drop on the tile is required (First Ten Angstrom, Model 200, or equivalent).

Hypodermic Syringe—A gas tight syringe is used, such as a 1-mL hypodermic syringe, equipped with a No. 27 blunt tipped stainless steel needle, capable of providing 100 to 200 drops from 1 mL.

Reagents and Materials

Water—Type II reagent water (distilled) in accordance with ASTM Specification D1193-99. Diiodomethane (99+% purity).

Positioning of Substrates

The tile is not to be touched with the fingers or is not contaminated in any other way during the positioning on goniometer stage.

Procedure

The tile is tested in a constant temperature (73° F.±2° F.) and humidity environment (50±5% relative humidity). The goniometer is set up and the stage is leveled according to the manufacturer's instructions. The contact angle is measured for each discrete droplet of water and diiodomethane on the tile as described in ASTM D7334 or the manufacturer's literature for the instrument being used. The tile is positioned to deposit a drop without visible distortion of the drop shape due to movement. The tip of the hypodermic needle is set at the distance from the surface recommended by the manufacturer of the instrument (3 mm (⅛ in.) and a drop of test liquid 5 μL in size is deposited on the tile. The drop size is controlled to ±0.1 μL.

Contact Angle

The camera or video device is focused so that the image of the drop can be captured. Two measurements are made (one on each drop edge) for each of two drops (water and diiodomethane) on the tile using commercial software designed to extract contact angles from movies or images. For example, First Ten Angstrom software version 2.1, build 363, can be used or equivalent. If the contact angles on two edges are different by more than 4°, the values are eliminated and the test is repeated. The measurement is repeated 5 more times on new droplets. The contact angle for the tile is reported as the average of the six angles measured for each side.

High Speed Video Imaging.

The image acquisition speed captures at least 10-20 images from the time the drop hits the surface to the time it cannot be resolved from the surface of the sample. A capture rate of 900 images/s is used. The software described above extracts the contact angles from the video feed. The droplet volume is also calculated using the same software under the sessile volume. The contact angles are plotted with the sessile volume plots. Enough time is allowed for the drop to wet out to equilibrium. However, in highly absorptive systems, the drop absorbs into the material before equilibrium is achieved. In such cases in which the drop rapidly (<0.2 s) absorbs into the substrate, the video is progressed until 2% of the volume of the drop absorbs into the substrate. The contact angle is recorded at that time point. This might mean that the first resolved image in extremely fast absorbing systems if the second image shows more than 2% volume loss.

Surface Energy Method:

Calculation

The Owens-Wendt-Kaelble equation:

$${σ}_{lg}^{T} \frac{( cos θ + 1 )}{2} = {( {σ}_{lg}^{D} {γ}_{sg}^{D} )}^{1 / 2} + {( {σ}_{lg}^{P} {γ}_{sg}^{P} )}^{1 / 2}$$

where:
θ=the average contact angle for the test liquid on the test specimen,
σ_{lg}^{T}=the total surface tension of the test liquid in dyn/cm
σ^{D} and σ^{P}=the dispersive and polar components of the liquid surface tension, respectively, also in dyn/cm.
σ_{sg=}the total surface energy of the test substrate in dyn/cm
σ^{D} and σ^{P}=the dispersive and polar components of the test substrate, respectively, also in dyn/cm.

| | Surface Tension (σ_{lg}) (dyn/cm) | | |
|---|---|---|---|
| Solvent | Nonpolar | Polar | Total |
| Diiodomethane | 50.8 | 0 | 50.8 |
| Water | 21.8 | 51.0 | 72.8 |

The Owens-Wendt-Kaelble equation is simplified to the following when a dispersive (nonpolar) solvent such as diiodomethane is used:

$${σ}_{lg}^{T} \frac{( cos θ + 1 )}{2} = {( {σ}_{lg}^{D} {γ}_{sg}^{D} )}^{1 / 2}$$

The dispersive (nonpolar) component of surface energy (σ^{D}_{sg}) is determined. Surface tension properties for diiodomethane are known and included in the table above. The contact angle is experimentally determined using the method delineated above.

Upon inserting the calculated dispersive component of surface energy (σ^{D}_{sg}) for the substrate into the Owens-Wendt-Kaelble equation delineated above and using the contact angles determined for water, the polar component of surface energy (σ^{P}_{sg}) of the substrate is determined because the surface tension properties for water are known and included in the table above. The dispersive component (σ^{D}_{sg}) of the substrate is determined with diiodomethane as explained above.

Thermodynamic Parameters

Thermodynamic parameters are calculated by inserting surface energy components into the following equations of state:

Spreading Coefficient:

The spreading coefficient (S) is determined by de Gennes:

S=γ_{sg}^{T}−σ_{lg}^{T}−σₛₗ

where σₛₗ is the interfacial tension
Reference: de Gennes, P.-G., Reviews of Modern Physics (1985), 57, 827-863

Interfacial Tension:

The Owens-Wendt equation of state is used to determine interfacial tension (σₛₗ):

σₛₗ^{T}=γ_{sg}^{T}+σ_{lg}^{T}−2(σ_{lg}^{D}γ_{sg}^{D})^{1/2}−2(σ_{lg}^{P}γ_{sg}^{P})^{1/2}
Reference: D. K. Owens and R. C. Wendt, Journal of Applied Polymer Science (1969), 13, 1741-1747.

Work of Adhesion:

The work of adhesion (W) using the Dupré equation of state:

W=γ_{sg}^{T}+σ_{lg}^{T}−σₛₗ
Reference: A. Dupré, Theorie Mechanique de la Chaleur; Gauthier-Villars: Paris, 1869; pp 36W.

D. Method of Determination of Sebum Removal Achieved by Hair Cleansing and Refreshing Method using a Kit of Leave-On Composition and a Substrate

### Method of Preparation of Sebum-Treated Hair Samples

a. Treatment of Hair Switches with Artificial Sebum

An amount of 144 μg of artificial sebum (table 3 above) is spread on a cotton pad using a micropipette. The area in which the absorbed sebum resides is approximately 4 cm×4 cm. The sebum is transferred from the cotton pad onto a hair switches by rubbing the cotton pad onto the hair switch, moving from the top to bottom of hair switch. The transfer movement is repeated 10 times to ensure that more than 90% of the sebum is transferred to the hair switches. The transfer is confirmed by gravimetric method. For each data generated, three switches are prepared and measured.

b. Cleansing Process using a Substrate

The leave-on aqueous cleansing composition is applied by spraying on the hair switch treated with the sebum. The dosage of the composition applies is 0.05 g of the leave-on composition per g of hair (NOTE). The hair switch is then left for 10 seconds at room temperature to partially dry. After this period, the hair switch is massaged with a substrate for 30 seconds. The hair switch is then allowed to air dry for at least 1 hour before determining the sebum content. NOTE: In several examples, a dose of 0.1 g of the composition is used per 1 g of hair. The data in the Examples table indicates the dosage.

Determination of Sebum Amount (Method of D1)

The hair switch which is treated with artificial sebum, as described above, is placed on an aluminum foil on a flat surface. The total amount of sebum on the hair switch (before the cleansing process) is measured using Courage & Khazaka Sebumeter® SM 815. A Sebumeter (SM 815 from Courage-Khazaka) measurement is based on sebum spot photometry. The mat tape of the Sebumeter® SM 815 is brought into contact with hair or skin. It becomes transparent in relation to the sebum on the surface of the measurement area. Sebumeter is used to measure the total sebum amount at seven different points along the length of the hair switch. Both sides of hair switches are measured, making a total of 14 readings that are averaged. After the cleaning process using the substrate, the total sebum on the hair switch is measured again. Each experiment is repeated with 3 hair switches. The unit of total sebum measured using sebumeter is μg/cm². The standard deviation of total sebum measured is less than 5 μg/cm².

E. Evaluation of Clean Feel and Clean Appearance Achieved by Hair Cleansing and Refreshing Method using a Kit of Leave-On Composition and a Substrate (Method E1)

The air dried hair switches from the previous method (method D paragraph b) are rated by ten expert graders in terms of (a) clean (non-greasy) appearance and (b) feel based on a 5-point scale, 5 being the best clean (no greasy) and 1 being the worst clean (very greasy).

F. Method of Determination of Sebum Removal Achieved by Hair Cleansing Method using an Aqueous Pre-Wash Composition Followed by Shampooing

### Method of Preparing Sebum-Fluorescence Dye

An amount of 20 g of artificial sebum is mixed with 0.03 g of Tinopal B (Benzoxazole, 2,2′-(2,5-thiophenediyl) bis[5-(1,1-dimethylethyl)], from BASF). The mixture is prepared in a brown vial to prevent light exposure and heated to 54° C. using water bath to melt and mix the components. Artificial Sebum Composition is prepared by adding materials mentioned in below table and then heated to 54° C. using water bath, to make it a uniform mixture.

Method of Treating Hair with Aqueous Pre-Wash Composition (Method F1)

An amount of 0.20 g of a sebum-fluorescence dye mixture is applied and massaged onto hair switch onto natural virgin brown hair switches weighing 4.0 g via a syringe (dosage 0.05 g of sebum-fluorescence per g of hair). Immediately afterwards, an image of the hair switch is acquired using a digital single-lens reflex camera with parallel polarizers (image at t0) under 256 nm UV light having power of 8 W. A quantity of 0.4 g of the aqueous pre-wash composition is then applied, spread on hair switch and is left on the hair switch under 25° C. and 50% relative humidity for 30 minutes. Then, the hair is wetted with water and 0.4 g of shampoo is applied (dosage 0.1 g of shampoo per g of hair). The shampoo is massaged into the hair for 10 seconds and rinsed with deionized water for 10 seconds at a flow rate of 20 ml/minute. The hair switch is then allowed to air dry and images are taken using the same camera under the conditions described above (image at tw). The hair switch in this case is also assessed by expert graders, as described below. The area of the image occupied by blue color intensity light due to sebum fluorescence mixture is analyzed (selecting the entire hair switch) using 2D projection. For this analysis Java-based image processing program is used. Then, the mean projected area is determined for the hair switch at t0 (At0) and for the hair at tw (Atw) and the sebum removal is calculated using the equation given below. Each experiment is repeated with three hair switches and the results are averaged. The percent sebum removal is calculated using below equation: % Sebum Removal=100×(Atw/At0). The standard error of sebum removal is less than 10%.

G. Evaluation of Clean Feel and Clean Appearance Achieved by Hair Cleansing Method using an Aqueous Pre-Wash Composition Followed by Shampooing (Method G1)

The air dried treated hair switches are rated by ten expert graders in terms of clean (non-greasy) appearance and feel based on a 5-point scale, 5 being the best clean (no greasy) and 1 being the worst clean (very greasy).

### Examples and Compositions

The following examples illustrate embodiments of the invention described herein. The exemplified leave-on aqueous cleansing compositions can be prepared by conventional methods. It will be appreciated that other modifications of the compositions within the skill of those in the art can be undertaken without departing from the spirit and scope of this invention. All parts, percentages, and ratios herein are by weight, unless otherwise specified. Some components may come from suppliers as dilute solutions. The amount stated reflects the weight percent of the active material, unless otherwise specified.

### Methods of Making Leave-On Aqueous Cleansing Compositions

The 1,2-diol and solid particle are added into the leave-on aqueous cleansing composition neat or as pre-emulsion. In the latter case, an emulsifier is used to make the pre-emulsion. The compositions exemplified in the example tables, the 1,2-diol(s) is added into the leave-on aqueous cleansing composition as a pre-emulsion. The pre-emulsion making process is the making of step a below.

A non-limiting example of a method of making the leave-on aqueous cleansing composition comprises the following steps:
a. Mixing all oil-soluble components (including the 1,2-diol and solid particles) in a vessel. Heating of the components may be needed in order to allow the components to be melted;
b. Mixing all water-soluble components in a separate vessel, adjusting the temperature of the contents of the vessel to the same temperature as the oil phase of step a;
c. Combining the oil phase and the aqueous phase under a high shear mixer, such as a Turrax mixer (supplied by IKA);
d. Adding the thickener/rheology modifier (Sepigel 305 in some of the examples), if required, into the mixture of step c, applying high-speed mixing for 2-5 minutes until a uniform mixture is obtained.

### Compositions

TABLE 4
Examples of Aqueous Cleansing Compositions

| | Pre-Wash | | | |
|---|---|---|---|---|
| | Treatment | Comparative | Comparative | Comparative |
| Examples | Control | Ex. I | Ex. II | Ex. III |
| Ingredient | (wt./wt.) % | (wt./wt.) % | (wt./wt.) % | (wt./wt.) % |
| Distilled Water | QS | QS | QS | QS |
| Polyacrylamide & C13-14 Isoparaffin & | 0.75 | 0.75 | 0.75 | 0.75 |
| Laureth-7 (Sepigel 305) | | | | |
| Perfume | 0.2 | 0.2 | 0.2 | 0.2 |
| 1,2-Decanediol | 0 | 1 | 0 | 0 |
| Cellulose | 0 | 0 | 1 | 0 |
| Zinc carbonate | 0 | 0 | 0 | 1.6 |
| Hydrophobic silica | 0 | 0 | 0 | 0 |
| 1,2-Dodecanediol | 0 | 0 | 0 | 0 |
| Laureth-7 (emulsifier) | 0.2 | 0.2 | 0.2 | 0.2 |
| PEG-100 Stearate (emulsifier) | 0.2 | 0.2 | 0.2 | 0.2 |
| Cetearyl glucoside & Cetearyl alcohol | 0.2 | 0.2 | 0.2 | 0.2 |
| Methyl paraben | 0.2 | 0.2 | 0.2 | 0.2 |
| Propyl paraben | 0.15 | 0.15 | 0.15 | 0.15 |
| Benzyl alcohol | 0.4 | 0.4 | 0.4 | 0.4 |
| Phenoxyethanol | 0.4 | 0.4 | 0.4 | 0.4 |
| % Sebum Removal at dose of 0.10 g of | 78 | 90 | 72 | 81 |
| composition for 1.0 g of hair | | | | |
| Note b, Method F1 | | | | |
| Clean Feel Rating at dose of 0.10 g of | 2 | 3.5 | 2 | 3 |
| composition for 1.0 g of hair | | | | |
| (5 scale rating; 5 is the highest clean | | | | |
| feel and 1 is the lowest) | | | | |
| Note b, Method G1 | | | | |
| Clean Look Rating at dose of 0.10 g of | 2 | 3.8 | 3.2 | 3 |
| composition for 1.0 g of hair | | | | |
| (5 scale rating; 5 is the highest clean | | | | |
| look and 1 is the lowest) | | | | |
| Note b, Method G1 | | | | |
| | Comparative | Comparative | Comparative | |
| Examples | Ex. IV | Ex. V | Ex. VI | Ex. VII |
| Ingredient | (wt./wt.) % | (wt./wt.) % | (wt./wt.) % | (wt./wt.) % |
| Distilled Water | QS | QS | QS | QS |
| Polyacrylamide & C13-14 Isoparaffin & | 0.75 | 0.75 | 0.75 | 0.75 |
| Laureth-7 (Sepigel 305) | | | | |
| Perfume | 0.2 | 0.2 | 0.2 | 0.2 |
| 1,2-Decanediol | 0 | 0 | 1 | 1 |
| Cellulose | 0 | 0 | 1 | 0 |
| Zinc carbonate | 0 | 0 | 0 | 1.6 |
| Hydrophobic silica | 0.5 | 0 | 0 | 0 |
| 1,2-Dodecanediol | 0 | 1 | 0 | 0 |
| Laureth-7 (emulsifier) | 0.2 | 0.2 | 0.2 | 0.2 |
| PEG-100 Stearate (emulsifier) | 0.2 | 0.2 | 0.2 | 0.2 |
| Cetearyl glucoside & Cetearyl alcohol | 0.2 | 0.2 | 0.2 | 0.2 |
| Methyl paraben | 0.2 | 0.2 | 0.2 | 0.2 |
| Propyl paraben | 0.15 | 0.15 | 0.15 | 0.15 |
| Benzyl alcohol | 0.4 | 0.4 | 0.4 | 0.4 |
| Phenoxyethanol | 0.4 | 0.4 | 0.4 | 0.4 |
| % Sebum Removal at dose of 0.10 g of | 86 | 85 | 76 | 97 |
| composition for 1.0 g of hair | | | | |
| Note b, Method F1 | | | | |
| Clean Feel Rating at dose of 0.10 g of | 3.5 | 3 | 2 | 4.5 |
| composition for 1.0 g of hair | | | | |
| (5 scale rating; 5 is the highest clean | | | | |
| feel and 1 is the lowest) | | | | |
| Note b, Method G1 | | | | |
| Clean Look Rating at dose of 0.10 g of | 3.5 | 3 | 1 | 4.5 |
| composition for 1.0 g of hair | | | | |
| (5 scale rating; 5 is the highest clean | | | | |
| look and 1 is the lowest) | | | | |
| Note b, Method G1 | | | | |
| | | | Comparative | Comparative |
| Examples | Ex. VIII | Ex. IX | Ex. X | Ex. XI |
| Raw Material | (wt./wt.) % | (wt./wt.) % | (wt./wt.) % | (wt./wt.) % |
| Distilled Water | QS | QS | QS | QS |
| Polyacrylamide & C13-14 | 0.75 | 0.75 | 0.75 | 0.75 |
| Isoparaffin & Laureth-7 | | | | |
| (Sepigel 305) | | | | |
| Perfume | 0.2 | 0.2 | 0.2 | 0.2 |
| 1,2-Decanediol | 1 | 0 | 0 | 1.0 |
| Zinc carbonate | 0 | 1.6 | 0 | 0 |
| Hydrophobic silica | 0.5 | 0 | 0 | 0 |
| 1,2-Dodecanediol | 0 | 1.0 | 1.0 | |
| Talc | 0 | 0 | 1.0 | 1.0 |
| Laureth-7 (emulsifier) | 0.2 | 0.2 | 0.2 | 0.2 |
| PEG-100 Stearate (emulsifier) | 0.2 | 0.2 | 0.2 | 0.2 |
| Cetearyl glucoside & Cetearyl | 0.2 | 0.2 | 0.2 | 0.2 |
| alcohol | | | | |
| Methyl paraben | 0.2 | 0.2 | 0.2 | 0.2 |
| Propyl paraben | 0.15 | 0.15 | 0.15 | 0.15 |
| Benzyl alcohol | 0.40 | 0.40 | 0.40 | 0.40 |
| Phenoxyethanol | 0.40 | 0.40 | 0.40 | 0.40 |
| % Sebum Removal at dose of | 93 | 90 | 68 | 76 |
| 0.10 g of composition for 1.0 g | | | | |
| of hair | | | | |
| Note b, Method F1 | | | | |
| Clean Feel Rating at dose of | 4.0 | 3.5 | 1.0 | 2.0 |
| 0.10 g of composition for 1.0 g | | | | |
| of hair | | | | |
| (5 scale rating; 5 is the highest | | | | |
| clean feel and 1 is the lowest) | | | | |
| Note b, Method G1 | | | | |
| Clean Look Rating at dose of | 3.8 | 3.8 | 3.2 | 2.0 |
| 0.10 g of composition for 1.0g | | | | |
| of hair | | | | |
| (5 scale rating; 5 is the highest | | | | |
| clean look and 1 is the lowest) | | | | |
| Note b, Method G1 | | | | |
| | Comparative | Comparative | Comparative | Comparative |
| Examples | Ex. XII | Ex. XIII | Ex. XIV | Ex. XV |
| Ingredient | Wt % | Wt % | Wt % | Wt % |
| Distilled Water | QS | QS | QS | QS |
| Polyacrylamide & C13-14 | 0.75 | 0.75 | 0.75 | 0.75 |
| Isoparaffin & Laureth-7 | | | | |
| (Sepigel,305) | | | | |
| Perfume | 0.2 | 0.2 | 0.2 | 0.2 |
| 1,2-decanediol | 0 | 0 | 0 | 1 |
| 2-hexyldecanol | 1 | 0 | 0 | 0.5 |
| Isoparaffin | 0 | 1 | 0 | 0 |
| Isododecane | 0 | 0 | 1 | 0.5 |
| Laureth-7 (emulsifier) | 0.2 | 0.2 | 0.2 | 0.2 |
| PEG-100 Stearate (emulsifier) | 0.2 | 0.2 | 0.2 | 0.2 |
| Cetearyl glucoside & Cetearyl | 0.2 | 0.2 | 0.2 | 0.2 |
| alcohol | | | | |
| Methyl paraben | 0.2 | 0.2 | 0.2 | 0.2 |
| Propyl paraben | 0.15 | 0.15 | 0.15 | 0.15 |
| Benzyl alcohol | 0.40 | 0.40 | 0.40 | 0.40 |
| Phenoxyethanol | 0.40 | 0.40 | 0.40 | 0.40 |
| % Sebum Removal at dose of 0.10 g | 88 | 90 | 86 | 97 |
| of composition for 1.0 g of hair | | | | |
| Method F1 | | | | |
| Clean Feel Rating | 3.0 | 3.5 | 3.5 | 4.5 |
| (5 scale rating; 5 is the highest clean | | | | |
| feel). | | | | |
| Note b Method G1 | | | | |
| Clean Look Rating | 3.8 | 3.8 | 4.0 | 4.5 |
| (5 scale rating; 5 is the highest clean | | | | |
| look). Note b | | | | |
| Method G1 | | | | |

Note b:
The measurement is performed using a method wherein the dose of the composition on hair is 0.10 g of the composition per 1 g of hair

Results from Table 4

The evaluation data shown in Table 4, utilize Methods F1 and G1. As described in the “Evaluation Methods” section, the cleansing regimen (for Methods F1 and G1) involves the application of the composition on sebum-deposited hair, followed by shampooing of the hair. Thus, the cleansing protocol involves the use of the compositions in a pre-wash/shampoo regimen. The data of Table show that the combination of sebum modifiers 1,2-diols and hydrophobic particles provides sebum removal, clean feel and clean appearance benefits. More specifically,
The data for Comparative Ex Pre-wash control of Table 4 correspond to sebum-deposited hair treated with a pre-wash composition that comprise neither a 1,2-diol nor hydrophobic particles, followed by shampooing.
The data for Comparative Ex. I, Ex. VI, Ex. V, Ex. X and Ex. XI of Table 4 correspond to sebum-deposited hair treated with a pre-wash composition that comprise 1,2-diol but no hydrophobic particles, followed by shampooing. Note that cellulose and talc consist of particles, but these not meet the requirements of the invention.
The data for Comparative Ex. III, Ex. IV of Table 4 correspond to sebum-deposited hair treated with a pre-wash composition that comprise no 1,2-diol but has hydrophobic particles, followed by shampooing.
The data for Examples VII, VIII, and IX of Table 4 correspond to sebum-deposited hair treated with pre-wash composition that comprise both 1,2-diol and hydrophobic particles, followed by shampooing. The compositions of these examples correspond to the inventive composition.

The data indicates that sebum-deposited hair that are treated with a pre-wash composition comprising both 1,2-diol and hydrophobic particles and then with a shampoo, show reduced sebum content and a better clean feel and appearance than the comparative examples.

TABLE 5
Examples of Methods of Cleansing Comprising Leave-on Composition and Substrate

| | | Comparative | Comparative | Comparative |
|---|---|---|---|---|
| | Control | Regimen | Regimen | Regimen |
| Examples of Regimen | Sample | Ex. A | Ex. B | Ex. C |
| Composition Example | None | None | Ex I | Ex I |
| Substrate Used (Yes/No) | No | Yes | No | Yes |
| Amount of Sebum Measured (μg/cm²) | 40 | 30 | 15 | 5 |
| Method D1 | | | | |
| Clean Feel | 1 | 1 | 3.5 | 4.5 |
| (5 scale rating; 5 is the highest clean | | | | |
| feel). | | | | |
| Note b, Method E1 | | | | |
| Clean Look Rating | 1 | 1 | 3.8 | 4.5 |
| (5 scale rating; 5 is the highest clean | | | | |
| look). | | | | |
| Note b, Method E1 | | | | |
| | | | Comparative | Comparative |
| | Regimen | Regimen | Regimen | Regimen |
| Examples of Regimen | Ex. D | Ex. E | Ex. F | Ex. G |
| Composition Example | Ex VII | Ex XIV | Comp Ex 2 | Comp Ex 2 |
| Substrate Used (Yes/No) | Yes | Yes | No | Yes |
| Amount of Sebum Measured (μg/cm²) | 2 | 1 | 20 | 15 |
| Method D1 | | | | |
| Clean Feel | 4.5 | 4.8 | 2 | 2.5 |
| (5 scale rating; 5 is the highest clean | | | | |
| feel). | | | | |
| Note b, Method E1 | | | | |
| Clean Look Rating | 4.5 | 4.8 | 2 | 2.5 |
| (5 scale rating; 5 is the highest clean | | | | |
| look). | | | | |
| Note b, Method E1 | | | | |

Note b:
The measurement is performed using a method wherein the dose of the composition on hair is 0.10 g of the composition per 1 g of hair

Results from Table 5

The evaluation data shown in Table 5, utilize Methods D1 and E1. As described in the “Evaluation Methods” section, the cleansing regimen (for Methods D1 and E1) involves the application of the aqueous composition on hair, followed by massaging of the hair with a substrate, which corresponds to the inventive kit and cleansing method. Table 2 involves compositions that are provided in Table 1, but they are used in a leave-on/substrate cleansing regimen, which is the inventive method. More specifically,
a. The data for Control sample of Table 5 correspond to sebum deposited hair switch in which no other treatment has been performed.
b. The data for Comparative Regimen Ex. A of Table 5 correspond to sebum-deposited hair switch in which no composition is applied. The hair switch is only massaged using a substrate.
c. The data for Comparative Regimen Ex. B of Table 5 correspond to sebum-deposited hair switch in which a leave-on aqueous cleansing composition is applied, comprising both a 1,2-diol and hydrophobic particles, and the switch is not massaged using a substrate.
d. The data for Comparative Regimen Ex. C of Table 5 correspond to sebum-deposited hair switch in which a leave-on aqueous cleansing composition is applied, comprising a 1,2-diol but not hydrophobic particles, and the switch is then massaged using a substrate.
e. The data for Regimen Ex. D and Regimen Ex. E of Table 5 correspond to sebum-deposited hair switch in which a leave-on aqueous cleansing composition is applied, comprising both a 1,2-diol and hydrophobic particles, and the switch is then massaged using a substrate. This regimen corresponds to the inventive kit and method of cleansing the hair and the scalp.
f. The data for Comparative Regimen Ex. F of Table 5 correspond to sebum-deposited hair switch in which a leave-on aqueous cleansing composition is applied, comprising neither a 1,2-diol, nor hydrophobic particles, and the switch is not massaged using a substrate.
g. The data for Comparative Regimen Ex. G of Table 5 correspond to sebum-deposited hair switch in which a leave-on aqueous cleansing composition is applied, comprising neither a 1,2-diol, nor hydrophobic particles, and the switch is then massaged using a substrate.

The inventive Regimens (Ex. D and Ex. E) show improved ability to remove sebum from the hair and show better clean fell and clean appearance than the comparative regimen examples.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of Embodiments of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A kit for cleansing and refreshing the hair and the scalp comprising:
a. a leave-on aqueous cleansing composition comprising
(1) from about 0.1 wt. % to about 12 wt. % of a 1,2-diol having a carbon chain with a length of more than 8 carbons;
(2) from about 0.1 wt. % to about 10 wt. % of a solid particle, wherein
(a) the interfacial tension between the solid particle and sebum is from about 5 to about 18 dyn/cm;
(b) the sebum exhibits spreading coefficient on the solid, which is greater than about 22 dyn/cm; and
(c) the work of adhesion of the sebum to the solid particle, which is greater than about 75 dyn/cm.
(3) an aqueous carrier; and
c. a substrate for partially removing the leave-on aqueous cleansing composition from the hair and the scalp.

2. A kit for cleansing and refreshing the hair and the scalp according to claim 1, wherein the leave-on aqueous cleaning composition of claim 1 further comprises an emulsifier.

3. A kit for cleansing and refreshing the hair and the scalp according to claim 2, wherein the emulsifier is nonionic.

4. A kit for cleansing and refreshing the hair and the scalp according to claim 3, wherein the emulsifier is selected from the group consisting of PEG-100 stearate, laureth-7 and mixture thereof.

5. A kit for cleansing and refreshing the hair and the scalp according to claim 1, wherein the leave-on aqueous cleaning composition of claim 1 further comprises a polymeric rheology modifier.

6. A kit for cleansing and refreshing the hair and the scalp according to claim 1, wherein the leave-on aqueous cleaning composition of claim 1 further comprises one or more solvents.

7. A kit for cleansing and refreshing the hair and the scalp according to claim 6, wherein the solvent is selected from the group consisting of 1,3-butanediol, isopropyl myristate, 2-hexyldecanol, propylene glycol, PEG-3 glyceryl cocoate, PEG-7 glyceryl cocoate, isododecane, isoparaffin C11-12 and mixtures thereof.

8. A kit for cleansing and refreshing the hair and the scalp according to claim 1, wherein the solid particle of the leave-on aqueous cleaning composition is selected from the group consisting of zinc carbonate, hydrophobically-modified silica, hydrophobically modified clay, zinc oxide, polyethylene powders, polypropylene powders, polystyrene powders, calcium silicate, nylon, boron nitride, mica, zeolite, cyclodextrins, fumed silica, synthetic clays, fluorocarbon resins, polypropylene modified starches of cellulose acetate, particulate cross-linked hydrophobic acrylate or methacrylate copolymers and mixtures thereof.

9. A kit for cleansing and refreshing the hair and the scalp according to claim 1, wherein the 1,2-diol of the leave-on aqueous cleaning composition is selected from the group consisting of 1,2-dodecnediol, 1,2-decanediol, 1,2-octadecanediol and mixtures thereof.

10. A kit for cleansing and refreshing the hair and the scalp according to claim 1, wherein the substrate is made from fibers or nonwovens.

11. A kit for cleansing and refreshing the hair and the scalp according to claim 1, wherein the substrate is made from the group consisting of cellulose, keratin, wool, cotton, jute, linen, flax, acetate, acrylate, cellulose ester, polyamide, polyester, polyolefin, polyvinyl alcohol, or polyurethane and mixtures thereof.

12. A kit for cleansing and refreshing the hair and the scalp according to claim 1, wherein the kit is sold as a combination of a spray dispenser comprising the leave-on aqueous cleansing composition and a set of substrates.

13. A method of cleansing and refreshing the hair and/or the scalp comprising the steps of
(a) applying a leave-on aqueous cleansing composition to the hair and the scalp, and
(b) partially removing the leave-on aqueous cleansing composition from the hair and the scalp using a substrate,
wherein the leave-on aqueous cleansing composition comprises
(1) from about 0.1 wt. % to about 12 wt. % of a 1,2-diol having a carbon chain with a length of more than 8 carbons;
(2) from about 0.1 wt. % to about 10 wt. % of a solid particle, wherein
(a) the interfacial tension between the solid particle and sebum is from about 5 to about 18 dyn/cm;
(b) the sebum exhibits spreading coefficient on the solid, which is greater than about 22 dyn/cm; and
(b) the work of adhesion of the sebum to the solid particle, which is greater than about 75 dyn/cm.
(3) an aqueous carrier.

14. A method of cleansing and refreshing the hair and the scalp according to claim 13, wherein the application of the leave-on aqueous cleansing composition onto the hair and/or scalp is performed by spraying the composition onto the hair and scalp.

15. A method of cleansing and refreshing the hair and the scalp according to claim 13, wherein the application of the leave-on aqueous cleansing composition is performed by spraying the composition onto the substrate and then transferring and spreading the composition onto the hair and/or the scalp using the substrate.
